(19) United States
(12) Patent Application Publication
(10) Pub. No.: US 2004/0254500 A1
(43) Pub. Date: Dec. 16, 2004

(54) Device and method for collecting, transporting and recovering low molecular weight analytes in saliva

(76) Inventor: Allan D Pronovost, San Diego, CA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(21) Appl. No.: 10/484,315

(22) PCT Filed: Jul. 18, 2002
(86) PCT No.: PCT/US02/22843

Related U.S. Application Data
(60) Provisional application No. 60/305,888, filed on Jul. 18, 2001.

(51) Int. Cl. A61B 5/00

(52) U.S. Cl. 600/584

(57) ABSTRACT

This invention relates to low molecular weight analyte sequestration, storage, transport and recovery device and methods for its use with saliva samples in the detection of glucose and other low molecular weight analytes. A stimulated or non-stimulated saliva sample is contacted with a matrix (16) disposed within a container (12) that adsorbs glucose, absorbs water and excludes molecules above a certain size when hydrated. After hydration of the matrix by the saliva sample, the glucose is both adsorbed to the matrix, and migrates into the cavities within the matrix, whereas microbes such as bacteria and larger molecules such as proteins cannot. A membrane (18) keeps the matrix (16) in the bottom of the container (12). The sample is then transported to a location where the glucose will be eluted from the matrix and assayed.

### FIELD OF THE INVENTION

This invention relates to low molecular weight (MW) analyte sequestration, storage, transport and recovery devices and methods for use with saliva samples, with specific application to glucose.

### BACKGROUND OF THE INVENTION

For glucose detection, blood, and less often urine, are the preferred bodily fluids from which glucose testing is done. Glucose detection is conducted either for “monitoring” purposes in type 1 and 2 diabetics, diagnosis of diabetes, or for screening of individuals for routine medical purposes. The concentration of glucose in blood can range from a low of 40 to >800 mg/dL depending upon the fasting conditions and relative disease status of the individual. Urine produces about 0.5 g of glucose every 24 hours. Collection of blood or urine samples for glucose detection is either unpleasant, inconvenient or invasive.

It would be desirable to have a reliable method of detecting glucose from another type of bodily fluid that can be collected less invasively and more conveniently. One potential bodily fluid that contains glucose is saliva; however, saliva contains approximately 100-fold less glucose (0.4−4.0+mg/dL) than blood. Blood detection technology at current blood or urine sensitivity thresholds cannot be directly applied to saliva detection, as it is known not to be sensitive enough.

In addition, saliva contains a variety of components that will adversely affect detection of glucose after collection in a saliva sample. This prevents deferred analysis of the sample to determine glucose levels at collection. Saliva is a viscous, sticky fluid, containing bacteria, cellular debris, and foodstuffs. The factors that can affect glucose and its levels in saliva include: degradation of glucose by enzymes; use of glucose as a metabolite by salivary microbes; adherence of glucose to mucins, polysaccharides, and proteinaceous molecules in saliva; and the inherent molecular instability of the glucose molecule itself, owing to isomerization and other intramolecular variations. Glucose exists in a left and right form, the ratio of which can vary spontaneously; it converts, depending upon pH and ionic strength, to isomeric forms such as fucose and mannose, and it changes structural form based on rotation around anomeric carbon 2.

It would be desirable to have a device for collecting and storing the glucose from a mixed whole saliva sample that reduces or eliminates the detrimental effects of the other components of saliva on glucose thereby preserving the glucose for assay at a later time. Preferably, this device should be easy to use.

Various patents that describe the collection of saliva for assay and/or transport to a facility where it will be assayed, have been described. A variety of very simple, non-analyte-participatory, collection materials have also been used to facilitate collection of saliva out of the mouth with subsequent expression of the saliva by squeezing or plunging or some other mechanical means. Non-analyte participating means that the collection materials do not interact with the analyte or its preservation. These include bite-size sponges (U.S. Pat. No. 5,211,182), pads (U.S. Pat. No. 5,573,009), swabs (U.S. Pat. No. 5,026,521), & filter paper (U.S. Pat. No. 5,260,230). These physical methods of manipulation require the saliva fluid be wrung out from the pads without effect or benefit to the analyte being measured. They merely serve to hold the fluid including the analyte and all other components found in saliva until squeezed out.

The use of salivary gland stimulation to facilitate secretion of analytes from salivary glands for detection has been known for years with application to a variety of analytes, including glucose. For reference refer to [1, 2, 3, 4 and 5].

For all low molecular weight analytes that cross the salivary gland/blood vascular border, there is a 20 to 30 minute delay in the appearance of analytes such as glucose in saliva as compared to blood. As concerns glucose, there is also a narrower and truncated dynamic range for glucose in saliva, verses blood. Recent ingestion of food (<2 hours) may also contribute to glucose readings in salivary samples. One of the best uses for glucose detection in whole mixed saliva is for qualitative screening, after 2-8 hours of fasting, in addition to application to real-time monitoring or diagnosis. Qualitative screening involves identification of those patients with salivary glucose levels above a finite threshold (i.e. >0.4 mg/dL fasting).

### SUMMARY OF THE INVENTION

This invention provides a device and method for the collection, transport and recovery of glucose and/or other low molecular weight analytes in saliva samples, which can be provided by either stimulated or non-stimulated means. The device provides for simple saliva collection; sequestration of glucose from a saliva sample by partitioning within the sample; retention of glucose in a relatively stable and non-reactive form until analysis; easy transport and simple and immediate recovery of glucose upon delivery to the location where it will be analyzed. The method of this invention provides for the use of this device in various contexts.

In one aspect this invention provides a device for sequestering a low molecular weight analyte from a saliva sample and storing the low molecular weight analyte until it is assayed, comprising:

(a) a matrix, said matrix being able to absorb water from the sample and reversibly adsorb the low molecular weight analyte from the sample, and said matrix comprising cavities sized to accommodate the low molecular weight analyte within, and to exclude selected components of saliva, and

(b) a container, into which the saliva sample and the matrix may be placed.

In one embodiment of this device the low molecular weight analyte is glucose. In another embodiment the matrix is affixed to a solid support. In another embodiment, there is a permeable barrier in the device, said barrier functioning to partition the matrix from the saliva sample. In another embodiment the device additionally comprises a preservative. In another embodiment, the device additionally comprises a means for sealing the container. In yet another embodiment, the container has a marking on it that facilitates the collection of the saliva sample, as it indicates how much sample should be collected in the device. In another embodiment the device includes a plastic dropper, for transfer of saliva into the container.

In another embodiment, the device further comprises a second matrix in the container, said second matrix being layered on the top surface of the first matrix, and separated from said first matrix by a membrane, and said second matrix being able to absorb water from the sample, and said second matrix comprising cavities sized to accommodate the low molecular weight analyte within, and to exclude selected components of saliva.

In another embodiment, the device comprises:

(a) a matrix, said matrix being able to absorb water from the sample and reversibly adsorb the low molecular weight analyte from the sample, and said matrix comprising cavities sized to accommodate the low molecular weight analyte within, and to exclude selected components of saliva, and

(b) a solid support onto which said matrix is affixed.

The matrix that is used to adsorb the low molecular weight analyte can be any matrix that adsorbs the low molecular weight analyte on a molecular basis. Preferred matrixes are cross-linked agaroses, cross-linked zeolites such as alumino-silica adsorbent sieves, activated carbon, activated charcoal, or aluminum oxides (gel-aluminas), or cross-linked anionic or cationic polyamine or polyacrylamide flocculent material.

In another aspect, this invention is a method of method of sequestering a low molecular weight analyte from a saliva sample, comprising:

(a) providing:

(i) a matrix, said matrix being able to absorb water from the sample and reversibly adsorb the low molecular weight analyte from the sample, and said matrix comprising cavities sized to accommodate the low molecular weight analyte within, and to exclude selected components of saliva, and

(ii) a saliva sample, and

(b) contacting the matrix with the saliva with the matrix.

In another aspect this invention is a method of sequestering a low molecular weight analyte from a sample of saliva and storing it in a relatively stable form, which method comprising:

(a) providing:

(i) a matrix, said matrix being able to absorb water from the sample and reversibly adsorb the low molecular weight analyte from the sample, and said matrix comprising cavities sized to accommodate the low molecular weight analyte within, and to exclude selected components of saliva, and

(ii) a preservative; and

(b) contacting the matrix with the sample and preservative, such that the matrix becomes hydrated by the sample

In one embodiment, the low molecular weight analyte is glucose.

In another aspect this invention is a method of sequestering a low molecular weight analyte from a saliva sample and storing the low molecular weight analyte until detection, comprising:

(a) providing:

(i) a first matrix in the bottom of the container, said first matrix being able to absorb water from the sample and reversibly adsorb the low molecular weight analyte from the sample, and said first matrix comprising cavities sized to accommodate the low molecular weight analyte within, and to exclude selected components of saliva;

(ii) a second matrix in the container, said second matrix being layered on a top surface of the first matrix and separated from said first matrix by a membrane, and said second matrix being able to absorb water from the sample and said second matrix comprising cavities sized to accommodate the low molecular weight analyte within, and to exclude selected components of saliva, and

(ii) a preservative;

(b) introducing the sample into the container by applying the sample to the top surface of second matrix; and

(c) allowing the sample to hydrate the second matrix and then the first matrix.

In one embodiment, the low molecular weight analyte is glucose. In yet another aspect, this invention is a method of transporting a saliva sample from one location to a second location for detection, by sequestering and storing the glucose according to the methods of this invention.

In yet another aspect, this invention is method of assaying for a low molecular weight analyte in a saliva sample comprising:

(a) providing a matrix, said matrix being able to absorb water from the sample and reversibly adsorb the low molecular weight analyte from the sample, and said matrix comprising cavities sized to accommodate the low molecular weight analyte within, and to exclude selected components of saliva;

(b) contacting the matrix with the sample;

(c) removing liquid that is not absorbed into the matrix;

(d) releasing the low molecular weight analyte that is sequestered within the cavities of the matrix, and

(e) detecting the released low molecular weight a method of assaying a saliva sample for the level of a low molecular weight analyte.

In yet another aspect this invention is a method for assaying for a low molecular weight analyte in a saliva sample after it has been transported from a location of collection to a location of analysis.

### BRIEF DESCRIPTION OF THE DRAWINGS

FIG. 1 illustrates schematically the method of this invention using one embodiment of the device of this invention.

FIG. 2(a) and (b) are side elevation views of two alternative embodiments of the device of this invention.

FIG. 3 shows the correlation between saliva glucose levels with blood glucose levels.

FIG. 4 shows the results of a de-centralized facilitator study, correlating saliva glucose levels with blood glucose levels.

### DETAILED DESCRIPTION OF PREFERRED EMBODIMENTS

Glucose is a sugar molecule with a molecular weight of 180 daltons. Glucose, being soluble in an aqueous environment, essentially behaves as the aqueous solvent does. Water is the primary solvent in stimulated and non-stimulated whole saliva, although stimulated saliva comprises considerably more water. The device and methods of this invention utilize the principle that, as a low molecular weight analyte in water, glucose will in essence behave as water unimpeded by other components found in saliva. By the device and methods of this invention, glucose is sequestered within the saliva sample by treating it as a solute in water. Therefore, components in saliva that would degrade glucose (such as bacteria or enzymes), or otherwise interfere with its detection (such as glycoproteins which may bind it), are physically separated from glucose. Glucose can therefore be stably transported to, and subsequently detected in, another location. These principles are known in science with a variety of applications but have not been applied for the sequestration of glucose in saliva for the purpose of stable transport.

Although the device and methods disclosed herein refer specifically to their application for collecting, transporting and recovering glucose from saliva, those skilled in the art are aware that embodiments of the device and methods could also be applied to the collection, transportation and recovery of other low molecular weight analytes that are present in saliva. “Low molecular weight analyte” means an analyte generally considered in the art of development of assay development as either being: (a) considered as a chemical entity which can be assayed for by chemical means, such as glucose, alcohol, lactic acid, bilirubin, homocysteine, potassium or the like; or (b) in immunoassay art as having the features of a hapten, namely of lower relative molecular weight, with the requirement for conjugation to carrier proteins to elicit an amnestic antibody response for generation of antibodies, and the assay for which generally requires the use of a competitive format for bound/free separation, such examples including drugs of abuse, therapeutic drugs, steroids and hormones, thrombolytic cascade factors and the like. Analytes which fall into this category may be inorganic in nature, or organic and be composed of sugars, carbohydrate, lipids, peptides, polypeptides, glycoproteins, glycolipids, oligonucleotides or the like, generally with molecular weights less than, but not necessarily limited to, 40,000 daltons depending upon the nature of the analyte, the adsorbents used, and presence in alternative body fluids. The use of the device and methods disclosed herein for the collection, transportation and recovery of other low molecular weight analytes that are present in saliva, is intended to be included herein.

In addition to sequestering glucose by virtue of its movement as a solute in water, glucose may be sequestered from the other components of saliva through the principle of adsorption, which as used herein means the retention, or adhesion, of solid, liquid, or gas molecules, atoms, or ions by a solid or liquid. Absorption, as used herein, means the penetration of liquids into, or the soaking up of a liquid substance by, a matrix.

One method of this invention involves contacting a saliva sample with a matrix selected to adsorb glucose in solution, either directly or indirectly through its association with water, to absorb water from the saliva, and to exclude molecules above a selected size (molecular weight cut off limit; MWCO), when hydrated. Saliva sample, as used herein, means either a stimulated or non-stimulated mixed whole saliva sample, a non-whole saliva sample, or a non-mixed saliva sample. Mixed whole saliva as used herein, means oral fluid that comprises a combination of fluids from a number of sources including parotid, submandibular, sublingual, accessory glands, gingival mucosa and buccal mucosa. Non-mixed saliva means saliva produced and collected from a selected salivary gland or combination of selected salivary glands. Non-whole saliva means saliva collected by any means that renders the saliva process no matter the mechanism used, i.e., filtration, centrifugation, reagent addition, or the like. During or after hydration of the matrix by the saliva sample, the glucose is both adsorbed to the surface of the matrix, and migrates into the cavities or spaces within the matrix, whereas microbes such as bacteria, and larger molecules such as proteins, cannot. The glucose is therefore retained by adsorption and protected from degradation by size exclusion.

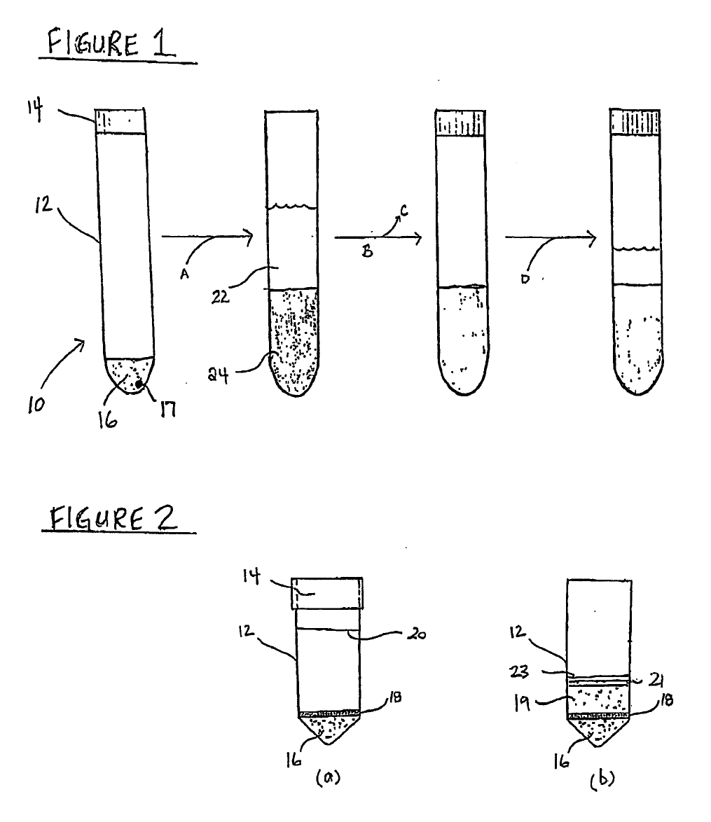

FIG. 1 demonstrates an embodiment of the device 10 of this invention. Device 10 comprises a container 12 with a sealing mechanism 14, containing a matrix 16. If the saliva sample will not be assayed immediately, a preservative 17 may be added to or mixed with matrix 16, or it may be a separate component of device 10, independent of matrix 16. In different embodiments the preservative may be a tablet that dissolves upon contact with saliva and mixes with the matrix, or it may be a powder that is mixed in with the matrix, or it may be dispersed or coated in a secondary media such as paper, glass, or plastic such that the coated media may be used by itself or in conjunction with other separation or detection means such as use as the sample absorption pad of a lateral flow strip used for detection of glucose by suitable means.

Container 12 as shown in FIG. 1 is shaped as a test tube. However, container 12 can be any of a number of shapes, including a test tube with a flat bottom or a vial. Preferably container 12 has a shape that is easy to use and transport. Additionally, the shape of container 12 ensures that, once the matrix is hydrated, it remains hydrated until the glucose therein is eluted. The size of container 12 may vary, as long as it is large enough to accommodate the volume of the saliva sample and the swollen matrix, together. Preferably, container 12 is as small as possible to achieve this objective, as a larger container would be wasteful of materials and space. In one embodiment, the volume within container 12 is between 0.5 and 20 ml.

Container 12 may be made of plastic, glass, acrylic or any other inert material. An inert material, as used herein, means a material that does not significantly interfere with the collection, transport, and recovery of glucose from the saliva sample, and therefore does not significantly affect the subsequent determination of the amount of glucose in the sample. Container 12 may be rigid or flexible. In one embodiment, container 12 is manufactured from a transparent material. This feature facilitates the collection of the sample by enabling the end-user to monitor the volume of sample in the container, and allows the end user to ensure that the matrix is being properly hydrated. However, it is to be understood that container 12 does not have to be transparent.

In one embodiment, container 12 has a marking on its surface that assists the end-user in determining whether sufficient saliva sample has been added to the container. This marking may be in any one of a number of forms, such as a line drawn on the container, a marked etched into the container, or a label positioned on the container.

As shown in FIG. 1, container 12 comprises sealing mechanism 14, that is used to seal the container before and/or after the saliva sample has been added. In the embodiment shown in FIG. 1, sealing mechanism 14 is a lid that threads onto container 12. Alternative means of sealing container 12, such as snap-on lids, or stoppers, are intended to be included herein. If container 12 is flexible, it may be sealed by a Zip-lock type of mechanism or by polymer welding.

Before addition of the saliva sample, container 12 can be sealed to avoid spillage of the matrix out of the container, or to ensure that the inside of the container remains sterile. However, if the various components of device 10 are to be assembled by the end user, then sealing of container 12 before addition of the saliva sample may not be necessary. For instance, the end-user may add matrix 16 to container 12 immediately before or immediately after the saliva sample is added, and therefore it may not be necessary to seal container 12 before that point.

After addition of the saliva sample, container 12 may be held upright if required until adsorption has been facilitated which may be from 0.5 to 30 minutes. Some adsorption media may adsorb analyte instantaneously as well. Container 12 can also be sealed to avoid spillage and evaporation of the saliva sample. However, if the assay for glucose occurs immediately after collection of the sample, without transport, sealing of the container is not necessarily required.

The sealing mechanism as shown in FIG. 1 is reversible, in that lid 14 can be secured onto, and then removed from, container 12. However, this is not necessary. The device may be designed to have different sealing mechanisms that are used before and after addition of the saliva sample. For instance, container 12 may have a snap-off portion or a pull-off portion, which is snapped off or pulled off and cannot be used again. The tube may be sealed after addition of the saliva sample by another means, such as a threaded lid or adhesive seal.

Matrix, as used herein, means a material in which something is enclosed or embedded, and more particularly means a material that comprises cavities, into which water, glucose and other small molecular weight molecules can migrate. As used herein, cavities means the spaces, pores or openings that are within the matrix. Matrix 16 has several functions, including: (a) adsorption of glucose; (b) absorption of water and smaller molecular weight components of saliva into the cavities; (c) exclusion of components of saliva above a selected size from its cavities, which components would degrade or bind glucose, and (d) allowing the recovery of glucose for assay purposes.

Matrix 16 may be a polymer with cavities formed by cross-linking of the monomeric units. The size of the cavities in matrix 16 after hydration should be sufficiently small to exclude selected components of saliva that would degrade or metabolize glucose, bind to glucose, or otherwise interfere with the measurement of the level of glucose in a particular saliva sample. These selected components would include, but not be limited to, proteins such as enzymes that degrade glucose, glycoproteins that bind glucose, and bacteria that would metabolize glucose. The preferred molecular weight cut off (MWCO) for matrixes that are useable in the method and device of this invention is about 1800 daltons, which would exclude proteins, glycoproteins and bacteria.

Matrixes that hydrate rapidly are preferred for use in this invention. Hydrate, as used herein, means the taking up or absorption of water from saliva. Rapid, as used herein means that the matrix will absorb a substantial amount of water from the saliva in between about 30 seconds to about 30 minutes. For example, when an agarose matrix is used, the matrix will hydrated within about 10 minutes. By way of explanation and not limitation, the more rapid the hydration, the sooner glucose will migrate into the cavities of the matrix, where it will be protected from larger molecular weight factors that would degrade, or bind to, it. Additionally the more rapid the hydration, the sooner glucose will be in contact with the matrix, where it will be adsorbed.

The matrixes should also adsorb glucose and allow the easy recovery of the glucose for assay purposes. Several matrixes are useful in this invention, including cross-linked agaroses, macroporous zeolites, alumino-silicate adsorbant sieves, activated carbon, activated charcoal, aluminum oxides (gel aluminas), or cross-linked anionic or cationic polyamine or polyacrylamide flocculent material (PAMS).

Cross-linked agaroses, prepared by cross-linking dextran with epichlorohydrin, are useful in the methods of this invention. They are available in a dried bead form gel that rapidly hydrates upon contact with aqueous solutions, such as saliva. Preferred are Sephadex® G-10, 15 and 25, in superfine, medium or coarse grade, which have a MWCO of 700, 1,500 and 5,000 daltons, respectively. As used herein, MWCO refers to the size, at or below which a molecule must be, in order to migrate into the cavities of the matrix. Other agarose matrixes that may be used include other Sephadex® resins such as Sephadex® G-50, and various Superdex®, Superose®, Sephacryl® resins, of a variety of MWCO's and grades (available from Amersham Biosciences).

In another embodiment, matrix 16 is comprised of a natural or synthetic composite of aluminosilicates, for example zeolites. Zeolites have a rigid three-dimensional crystalline structure with cavities of uniform size. There are a variety of naturally occurring zeolites (i.e. clinoptilolite), which are useful in the methods of this invention. Most crystalline structures however accommodate only small molecules such as cations. It is possible to obtain powdered (for coating) cross-linked aluminosilicate extruded rod stock, molded or pelleted material such as Zeolite-Y, Zeolite-Beta and ZSM-5 (available from Zeolyst International). All of the above zeolites are prepared to a controlled porosity with a MWCO limit of approximately 800 daltons to accommodate the glucose in saliva. Glucose adsorption is facilitated through cation displacement upon primary hydration. An alternative to natural zeolite is the gel aluminas, such as UOP International's (Illinois) Versal® aluminum oxides (Al₂O₃), which are synthetic adsorbents. Activated carbon (Snowblack Activated Carbon Co Ltd.), or activated charcoal (Sigma) may also be used.

In another embodiment, matrix 16 is flocculent anionic or cationic cross-linked polyamine or polyacrylamide (PAMS). PAMS suitable for the methods of this invention include Superfloc™ C-573, C-577, C-580, C-581 and C-582 (available from Cytec Engineered Materials).

Matrix 16 may be used in a variety of forms, such as a powder, a pellet, a rod, or as a coating to another media. For example, matrix 16 may be affixed, or otherwise attached or applied, reversibly or irreversibly, to a solid support such as a stick, rod, slide, wick, card, and the like, using techniques that are known to those skilled in the art. For example, the matrix may be coated onto a solid support. In one embodiment, the container is the solid support for the matrix, and the matrix is affixed to the container by being placed within the container.

FIG. 2(a) shows an alternative embodiment of device 10, which may be used when matrix 16 is in powder form. In this embodiment, container 12 has a conical base that holds matrix 16. In order to keep matrix 16 in the bottom of container 12, and in order to avoid disruption of the matrix when saliva is added, a membrane 18 is placed on top of matrix 16. Additionally, membrane 18 may serve as a gross pre-filter, to separate out or trap cellular debris or gross molecular weight components. Membrane 18 may be comprised of any of a number of inert materials, including: glass fiber; filter paper; PVC membrane filters; polypropylene; polyethersulfone, & RW (microporous polymer of cellulose ester formed around a polyester web) prefilters; nylon membrane; supported, unsupported and hydrophilized PTFE; quartz fiber filters; mixed cellulose esters filters; polyvinylidene fluoride membrane; dacron or rayon or polyethelene membranes; cellulose acetate membranes; cellulose nitrate membranes; nitrocellulose membranes, or the like.

This embodiment also demonstrates a fill line 20, which notifies the end user when a sufficient volume of saliva sample has been collected.

A preservative may be added to container 12 either before or after the saliva sample is added. Compounds contemplated as preservatives include anti-bacterial agents, anti-fungal agents, bacteriostatic agents, fungistatic agents, and enzyme inhibitors. These preservatives may be used alone or in combination. One useful preservative is 2-methyl-4-isothiazolin-3-one (ProClin; Rohm and Haas Company), and more particularly ProClin 200 at between 20-400 ppm, preferably 50 ppm. Other preservatives that may be used include sodium azide, benzoic acid, sorbic acid, and the salts thereof, thimerosal, phenyl mercuric acetate, Kathon (5-chloro-2-methyl-4-isothiazolin-3-one; Rohm and Haas Company), phenyl mercuric nitrate, ethyl alcohol and chlorhexidine gluconate and benzalkonium chloride, singly or in combination. These preservatives are used at about 0.01 to about 0.5% by weight. The preservatives may, among other things, inactivate enzymes and therefore act as an inhibitor, such as is accomplished by Pefabloc (Roche Applied Sciences), destroy proteins, kill bacteria or attenuate bacterial growth.

Having thus described device 10 of this invention, the method of its use will now be outlined in detail, referring again to FIG. 1. In the first step of this method, indicated by arrow A, a saliva sample 22 is introduced into container 12, preferably by expectoration. However other means of introducing the sample into the tube, such as collection in a secondary container such as a suction tube, or collection by an absorbent swab, are intended to be included herein.

Saliva sample 22 is either a stimulated or unstimulated saliva sample. Stimulation of saliva is accomplished by any of a number of means including: citric acid (1-200 mg, preferably 50 mg); sodium citrate (1-200 mg); potassium chloride (0.1-10% by weight); sodium chloride (0.1-10% by weight) and potassium tartrate (0.1-10% by weight). A saliva sample, either stimulated or unstimulated, of between 0.1 and 5 mL, preferably between 0.3 and 0.8 mL, is collected and used in the methods of this invention. Preferably, the sample will have a sufficiently large volume to at least hydrate all of matrix 16 in container 12, however less saliva can be used, with a corresponding reduction in yield.

After saliva sample 22 is introduced into container 12, or while it is being introduced into container 12, the sample is caused to come into contact with matrix 16, whereupon the matrix will become a hydrated matrix 24. With the exception of zeolites, which do not expand upon introduction of saliva, the volume of hydrated matrix 24 is greater than the volume of matrix 16, as demonstrated in FIG. 1. As the second container 12 in FIG. 1 also demonstrates, the volume of the saliva sample may be greater than needed to hydrate matrix 16. The hydration of matrix 16 is preferably done at room temperature, or about 20° C., however it may occur at a lower or higher temperature as long as the integrity of the matrix is maintained. If the assay for glucose is to occur immediately, the sample may be stored at room temperature, otherwise it may be stored at 4° C. or lower.

The hydration of matrix 16 is allowed to occur for about 0.5 to 30 minutes before assaying for glucose. The saliva sample in container 12 is then transported to the location where it will be assayed for glucose content, as indicated by arrow B of FIG. 1. This Figure demonstrates that transport occurs after hydration of the matrix has occurred, but it may be concurrent with it. The two may be held upright during this process. The sample is transported at ambient temperature, or between about freezing and 30° C., and preferably between freezing (about 0° C.) and 10° C.

Although the glucose is preserved in the sample after the matrix is hydrated, it is not infinitely stable unless stored at between −20° C. and −70° C. Therefore, transportation to the location of analysis is ideally accomplished overnight or up to about 3 days after sample collection, however it can be extended to about 5 to 90 days through the addition of stabilizers such as pleuronic F68 (BASF) or the like. Longer transportation times may be used if the sample is refrigerated, which may be preferred for sample collection locations that are distant from the location where the sample will be analysed. Preferably, all transportation will be done at an internal pack temperature of 10° C., to retard microbial growth.

After the sample is transported to the location where it will be analysed for glucose content, excess liquid is removed from container 12, as indicated by arrow C in FIG. 1. The excess liquid can be removed by any one of a number of ways, including decantation, aspiration or centrifugation and removal of the supernatant. Alternatively, the matrix and liquid may be transferred to a drip column, and the liquid removed by gravity or vacuum, or to spin column and the liquid removed by centrifugation through the column. Alternatively, container 12 may be designed initially to be a drip column, or spin column that can be centrifuged. Alternatively, if the adsorbant matrix is used, dispersed in or coated on another media, it may be employed as the sample pad collection component of a lateral flow strip. Membrane 18 is removed, either before or after removal of the excess liquid, but preferably after. Any means of removing the excess liquid from the hydrated matrix is intended to be included herein.

Depending upon the matrix used, there may or may not be a washing step before elution of the glucose. For example, for agarose matrixes, washing before elution is not necessarily required, whereas for zeolite matrixes a washing step is generally included before elution.

The glucose is then eluted from hydrated matrix 24 by bringing the hydrated matrix into contact with an elution solution 26 that will cause the adsorbed glucose to be released from the matrix. This step is indicated by arrow D in FIG. 1. In one embodiment solution 26 is a salt solution, for example saline, that causes the glucose to be eluted by reverse desalting. This solution can be used when the matrix is a cross-linked agarose. In another embodiment the glucose is eluted by reverse cation exchange, using a salt solution, such as KCl or NaCl, between about 0.1 to 40% by weight preferably in water, and preferably about 4% KCl or NaCl. This solution can be used when the matrix is a zeolite. In yet another embodiment, solution 26 is a reverse ionic solution, which is used to elute glucose from a matrix such as a flocculent anionic or cationic cross-linked polyamine or polyacrylamide. For example, if the matrix were anionic, such as Anionic PAM Emulsion 1036 (Magnifloc® flocculent; Cytec Inc.) solution 26 would be cationic, such as alkyl amine salts, quaternary ammonium salts, or the like, such as a 1% solution of tetramethlylammonium chloride or the like (Sachem, Inc.). As is apparent, the type of solution that can be used depends upon the type of hydrated matrix 24 used. Additionally, for any particular matrix a number of different solutions could be devised which operate upon the same principle of eluting the adsorbed glucose from the matrix. These alternative solutions are intended to be included herein. The release of glucose is relatively instantaneous, and for agarose matrixes takes about one minute, and for zeolites about 30 minutes and the solution into which glucose is eluted is chemically defined minimizing the possibility of degradation. Hence, there is only a small possibility that the glucose will be degraded or bound after elution, however glucose detection is preferably still done immediately after elution from the matrix. If detection will be delayed, additional preservative may be added to the solution, or the solution may be frozen or refrigerated.

Elution may be accomplished by adding solution 26 to container 12, mixing hydrated matrix 24 and solution 26 together, and allowing the matrix to settle again, after which solution 26 containing glucose can be aspirated or decanted. This method is a preferred method of eluting the glucose. Alternatively, settling of matrix 24 may be assisted by centrifugation. In another embodiment, solution 26 may be removed and collected by transferring both hydrated matrix 24 and solution 26 to a separate drip column, or a spin column that can be centrifuged. In yet another alternative, container 12 may be designed initially to be a drip column, or spin column that can be centrifuged. In the latter method, centrifugation of solution 26 through hydrated matrix 24, rather than mixing solution 26 and matrix 24 together, may accomplish elution of the glucose from hydrated matrix 24. Any means of bringing solution 26 into contact with hydrated matrix 24, and separating solution 26 from matrix 24, is intended to be included herein.

In one embodiment of this invention described above, the matrix is not a powder, but rather is rod stock material or pellet. From 0.5 to 2 gm of rod material is used for a saliva sample of 1-4 mL. Unabsorbed liquid is removed from the matrix by removing the rod material from container 12 into another container, or alternatively by removing excess liquid from the container 12. The rod material is then washed 1-2× with water, if required, and glucose is then eluted by inserting the rod material into solution 26, which may be in a different container, or by adding solution 26 to the container. The rod material may also be pulverized after removal of the excess liquid is accomplished, and before or during elution of the glucose. The elution progresses for generally between about 0.5 and 30 minutes. The rod material is then removed, or allowed to settle.

The volume of solution 26 to be used for elution of glucose can vary. Generally, the volume of solution 26 added to the sample is approximately equivalent to the saturation volume of the matrix used. Therefore, for example, if a gel holds 0.5 mL of liquid per gram, 0.5 mL of solution 26 will be added to the gel to elute the glucose.

After hydrated matrix 24 is separated from solution 26, solution 26 is analysed for glucose content by YSI or other means. For example, the inventors have used a YSI 2700 analyzer for glucose, which provides electrochemical detection of glucose using a Glucose Oxidase/Horseradish Peroxidase membrane sensor. The YSI 2700 can be used in a range of 0-9 gm/L glucose. Linear calibration curves for salivary glucose in the 0.25-4.0 mg/dL range were developed for stimulated whole saliva, and used to assign a value of glucose content, in samples analysed. Any glucose analyzer, from bench top to hand-held may be used, provided that it can accurately measure glucose at levels as low as 0.5 mg/dL.

Types of Retention

Direct Retention

The main form of adsorption and retention described has been based on the adsorption of analyte below a MWCO limit with exclusion of undesired material above the MWCO limit. This is followed by elution by ion exchange. This approach assumes use of a MWCO limit from 0 to 700 daltons (or some other desired upper limit) for the explicit purpose of all- or- none adsorption/exclusion. With this particular resin the use of adsorption below the lower MWCO limit of 0 is obviously not possible and this limits the application unless adsorbants with both non-zero lower and upper MWCO limits are used.

Selective Retention

Separation media for employing adsorption and elution are available at a variety of MWCO ranges with upper and lower limits. Separation media not operating at the lowest limit of retention offers a range of molecular weight retentions that can be selected depending upon application (molecular weight of the analyte). When an adsorbent has a MWCO range above zero, as example, from 1,500 to 3,000 daltons, analyte in this MWCO range will be selectively retained within the adsorbent based on the volume of fluid imbibed. For an absorbant with a MWCO range from 1500 to 3000 daltons, as example, material with a MWCO below the lower MWCO limit (1500 daltons) will pass through the adsorbent unaffected (unretained in what is referred to as the void volume). Material above the upper MWCO limit will not enter into the adsorbent and reside on the front surface of the adsorbent based on unidirectional fluid flow.

Selective retention can be used for several purposes:

(a) to selectively retain material in a defined MWCO range (eg. an analyte in the above example with a MWCO of 2,000 would be retained with elution by ion exchange at a later point);

(b) to selectively remove materials (interfering substances) above the lower limit of the MWCO range in an application wherein the analyte MW is below the lower MWCO limit cutoff, and said material below the lower limit of the MWCO range is allowed to flow unretained. Elution by ion exchange is not required in this latter application as the analyte passes through the adsorbant unimpeded in the void volume. Material in the MWCO range will be retained. This approach can be used in an all-in-one device for low molecular weight detection through the use of separately dispersed or coated adsorbents in the sample pad component of an integrated lateral flow strip detection means for glucose.

Broad Range Selective Retention

Adsorbents are available with broad MWCO limits (eg. 3,000 to 150,000 daltons and 30,000 to 1,000,000 daltons). Broad MWCO retention ranges not inclusive of zero allow for the retention of a broad range of molecular weight species of high molecular weight. The use of these broad range adsorbents would be of use to remove a broad range of molecular weight species as is found in saliva samples and could also be used if dispersed in or coated on the sample collection pad of an all-in-one device.

For sample collection, a 1 mL sample of saliva is added and allowed to adsorb. In this device, glucose, as analyte, flows through the Superose 12 unrestrained directly into the Sephadex G10 layer, and whereas all potentially interferent materials with a MW of 1,000-5,000,000 are actively retained in the Superose matrix. After transport to the lab, the top glass fiber membrane, the Superose 12 layer, and the lower glass fiber membrane are removed and discarded. Glucose is then eluted from the Sephadex layer as described above, free from all potential interfering substances.

In another embodiment using the same device as constructed above, one could apply its use to detection of lutenizing hormone of MW 30,000 daltons in urine or saliva containing potential interfering materials, wherein lutenizing hormone would be detected in the Superose 12 layer after elution and all potentially interfering materials with MW's below 1,000 would be retained by the bottom Sephadex layer, or potentially interfering materials with MW's above 5,000,000 daltons in the upper top glass fiber layer respectively.

Differential Retention

Combinations of adsorbents can be used to retain analytes, allow analytes to pass unimpeded or to remove unwanted material. Numerous combinations can be used, some of which are described herein.

Both dual and triple combinations can be used for different analytes of different MW. Adsorbents may be employed in layers in a tube, zones on a sample pad strip so as to facilitate use. The examples below assume sample fluid contact with layer 1, then layer 2, etc. as described below, in a unidirectional flow path. All combinations of adsorbants address variations on the main principle, either to retain and sequester or remove and avoid the desired or undesired species of interest whether it be analyte or interferent, and it is understood by those skilled in the art the examples below are included by reference.

Examples of how differential retention may be accomplished using layers or zones of matrix, are provided in Table 1.

TABLE 1
Differential Retention

| Target | Adsorbent | Layer | Layer MWCO | Final Analyte | Layer |
|---|---|---|---|---|---|
| Analyte | Layer | Type | Range | Position | Use |
| Low | 1 | Broad High | >3 K (to 3 M) | | Removal of all |
| (eg. <2,000) | | | | | unwanted high |
| | | | | | MW |
| | 2 | Low (to zero) | <3 K | X | Retention of |
| | | | | | anaalyte |
| | 1 | Highy | >50 K to 3 M | | Removal of |
| | | | | | unwanted |
| | | | | | high MW |
| | 2 | Medium | >3 K to 50 K | | Selected |
| | | | | | Remov al of |
| | | | | | unwanted |
| | | | | | high MW |
| | 3 | Low (to zero) | <3 K | X | Retention of |
| | | | | | analyte |
| Moderate | 1 | High | >15 K to 3 M | | Removal of |
| (eg. 12,000) | | | | | unwanted |
| | | | | | high MW |
| | 2 | Medium | >5 K to 15 K | X | Retention of |
| | | | | | analyte |
| | 3 | Low | <5 K | | Removal of |
| | | | | | low MW |
| | | | | | interferent |

The different types of retention disclosed above may be combined. For example, broad range selective retention may be combined with layering. This embodiment is shown in FIG. 2(b). To a collection tube (12), 2 adsorbants of varying MWCO range are layered. The bottom layer comprises 0.20 gm of a matrix (16), such as dry Sephadex G-10 powder (MWCO 0-700 daltons; Amersham), on top of which is layered a membrane (18), such as a piece of Whatman GF-B glass fiber membrane. On top of the first glass fiber membrane in the tube, is layered 0.20 gm of a second matrix (19), such as dry Superose 6 (MWCO range of 1,000-5,000,000 daltons; Amersham). A second membrane (21), such as a Whatman GFB glass fiber membrane is added on top to hold the assembly in place followed by dye cut, inert, polypropylene plastic mesh (23).

### EXAMPLES

1. Determination of Relative Adsorption.

Five types of matrix were tested in duplicate and compared for relative adsorption of glucose after elution. Each tube contained 0.3 g of matrix. A molar excess of stock glucose containing 10 mg/dL glucose was added to each sample to afford the maximum potential for adsorption without risk of nonsaturation. After elution from the matrixes, the modified YSI 2700 biochemical analyzer was used to determine the concentration glucose after dilution by elution.

Samples Tested:

Sample A: Sephadex G-10

Sample B: Sephadex G-15

Sample C: Sephadex G-25 coarse

Sample D: Sephadex G-25 medium

Sample E: Zeolyst CP814 (lot 01-12)

After addition of a molar excess of glucose solution, each sample was incubated at room temperature for approximately one hour to allow adsorption. Excess liquid was drawn off after one hour. Each tube containing matrix was washed 3× with deionized water to remove non imbibed glucose and one mL of elution solution (2% NaCl for the gels, 2% KCl for the Zeolyst) was added to each tube to elute the matrix contents. Samples were vortexed gently, allowed to settle for 15 minutes, and then eluates were tested for glucose content. Recorded in Table 2 below are the amounts of glucose detected after elution of the imbibed fluid from various matrix types into 1 mL of extraction fluid. These results show that all matrixes adsorb glucose which can then be eluted and detected. The matrixes vary in the relative amount of glucose imbibed.

TABLE 2
Concentration of Glucose in Eluant

| Sample | Concentration (mg/dL) |
|---|---|
| A1 | 4.06 |
| A2 | 3.79 |
| B1 | 6.21 |
| B2 | 6.22 |
| C1 | 4.15 |
| C2 | 4.78 |
| D1 | 3.92 |
| D2 | 4.19 |
| E1 | 2.80 |
| E2 | 2.84 |

3. Determination of Fluid Absorption Capacity of Various Matrixes

The approximate amount of liquid that is absorbed by the various matrixes was determined. One gel (Sephadex G-10) and 4 zeolyte types, each representing different grades of Zeolyst crystal, were tested. The zeolite types tested were: CBV 28014; CP 814; CBV 8014 and CBV 500.

Each sample tube contained 0.2 grams of each matrix. Each of the tubes was weighed to determine a base line value of the weight of the crystals plus the tube. Absorption of water was measured by the change in weight of the tube and matrix, after water was added, allowed to sit with the crystals for 2 hours, and then all excess fluid was removed. The average volume of water absorbed by 0.2 gm of matrix is shown below in Table 3.

TABLE 3
Volume of Water Absorbed by 0.2 gm of Various Matrixes

| Matrix | Average Volume of Water Absorbed |
|---|---|
| Sephadex G10 | 0.454 mL |
| CBV 28014 | 0.145 mL |
| CP 814 | 0.249 mL |
| CBV 8014 | 0.119 mL |
| CBV 500 | 0.132 mL |

The average volume of water that is absorbed represents the minimum sample volume required to completely hydrate the matrixes.

3. Determination of Whether Various Matrixes Inherently Contain Measurable Amounts of Glucose

Approximately 0.3 gm of each type of Zeolyst was placed in a tube with water for 30 minutes or 24 hours. After elution, the eluate was tested for glucose concentration. Each matrix was tested in triplicate. The YSI 2700 biochemical analyzer was used to test for glucose content. The results obtained, shown in Table 4, indicate that the matrixes do not contribute glucose to the measurement.

TABLE 4
Inherent Glucose Content of Matrixes

| | Control T = 30 Min | T = 24 hours |
|---|---|---|
| Sample Type | Concentration mg/dL | Concentration mg/dL |
| Sephadex 010 | 0.001 | 0.001 |
| CBV 28014 | 0.000 | 0.000 |
| CBV 28014 | 0.024 | 0.006 |
| CBV 28014 | 0.061 | 0.013 |
| CP 814 | 0.037 | 0.039 |
| CP 814 | 0.067 | 0.026 |
| CP 814 | 0.055 | 0.086 |
| CBV 8014 | 0.055 | 0.066 |
| CBV 8014 | 0.073 | 0.059 |
| CBV 8014 | 0.067 | 0.026 |
| CBV 500 | 0.104 | 0.026 |
| CBV 500 | 0.092 | 0.039 |
| CBV 500 | 0.085 | 0.059 |

4. Clinical Findings

Clinical confirmation of the procedure disclosed herein, using Sephadex G10 as matrix and Whatman GF-B as prefilter for collection and processing, was accomplished in 2 non-fasting studies, 3 controlled fasting studies and in a de-centralized facilitator study. All studies involved direct correlation with parallel blood samples. A total of 87 diabetics were studied in addition to 216 non-diabetics. All studies involved stimulation. Approximately 1 mL of saliva was collected for each sample, however the volume of saliva collected is not critical, except in so much that it must be sufficient to completely hydrate the matrix. All studies were conducted according to specific investigator approved protocols with IRB approval with the exception of the de-centralized facilitator study. All samples were analysed for glucose content using a YSI 2700 analyzer for glucose, a described above.

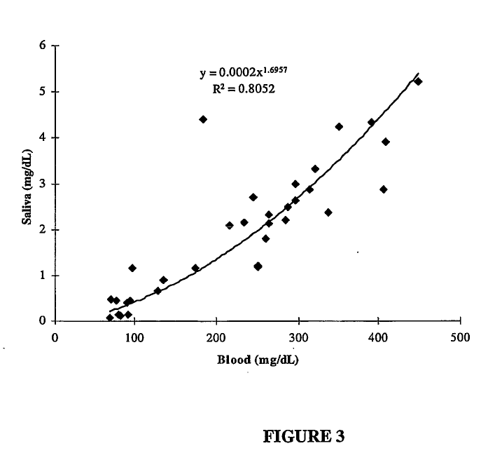

The non-fasting studies were conducted on 33 individuals, 42% of whom were diabetic and 58% non-diabetic. Patients abstained from food and drink for 2 hours prior to stimulation. Saliva glucose was correlated by power fit regression analysis with blood, as shown in FIG. 3.

Correlation was noted in the non-fasting population over the entire range from 70-500 mg/dL with R² values of >0.81 for either fingerstick or venipuncture blood/plasma measurements.

Non-fasting studies were followed by 3 successive fasting studies. Fasting studies were designed to assess, control, and identify any covariants. These successive fasting studies were conducted under IRB approval involving 193 patients. All patients met strict inclusionary criteria and had parallel fingerstick and venipuncture blood drawn. The protocol involved the sequential collection and processing of numerous saliva samples in conjunction with blood. Both blood and saliva determinations were performed on the same YSI analyzer. In addition, non-stimulated whole saliva samples were processed at the laboratory as back-up validation to the procedure conducted on site.

Data from the 3 studies were analyzed by correlation using linear regression in addition to Receiver-Operating Characteristic (ROC) analysis [6]. In addition, blood/saliva correlation, fingerstick vs. venipuncture correlation, and time after stimulation were assessed. ROC analysis facilitated the determination of screening assay cutoffs for both the 2-hour and 8-hour fasting procedures to maximize sensitivity and specificity for screening purposes.

Study population demographics were equally represented for gender, age, height, weight, and body mass index. Race averaged 85% of diabetics as white compared to 95% for non-diabetics

Covariants were not identified as necessary for or contributing to the determination of glucose in saliva. Measurement of glucose in saliva was found to differentiate diabetic and non-diabetic populations as well as blood was able to do. Results from all 3 studies indicated a blood correlation of >85% by linear regression analysis.

ROC analysis was used to select 2-hour and 8-hour saliva glucose values relative to blood glucose categories recommended by the American Diabetes Association (ADA) for screening [7,8]. Eight (8) hour fasting levels for normal (<110 mg/dL), borderline (≧110 <126 mg/dL), elevated (≧126 mg/dL) and 2-hour fasting levels (<200 mg/dL, normal; ≧200 mg/dL, elevated) were correlated to salivary glucose values. Optimal saliva glucose threshold concentrations were identified to differentiate upper and lower values relative to above blood/plasma cutoffs and were maximized for sensitivity and specificity for screening purposes through analysis of area under the curve. Data indicated that saliva glucose concentrations could be identified to afford a sensitivity approaching 99.99% (100%) with a specificity of 84% (the area under the curve accounted for 93%) for screening purposes using either the 8-hour or 2-hour fasting criteria.

To further confirm these findings a de-centralized facilitator study was initiated using 77 volunteers, which included 65 self-reporting normals, and 12 diabetics. All 8-hour fasting volunteers provided saliva samples using the Specimen Collection Kit with transport to the lab within 48 hours. Upon receipt at the lab all samples were frozen until tested. Results from the studies indicated an overall sensitivity of 99.99% (100%) and specificity of 96.6% by either the 8-hour or 2-hour classification criteria for saliva using the threshold cutoffs identified in the earlier study relative to ADA blood screening criteria.

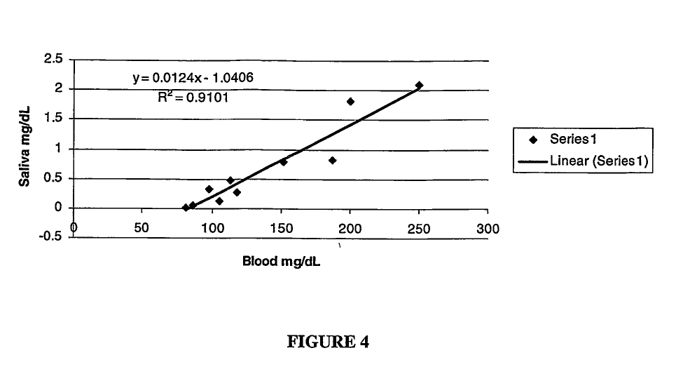

In addition, some volunteers reported fingerstick blood glucose values using an OTC monitoring device. For those volunteers reporting blood glucose values, the study results indicated a correlation of saliva to blood of 91% for all subjects and 91.3% for diabetics alone (R²0.9101 or 0.9130 respectively; FIG. 4).

### REFERENCES

The following references are cited in the application in brackets [ ], at the relevant portion of the application. Each of these references is incorporated herein by reference.

1. Baum (1993) Principles of saliva secretion, Acad. Sci. 694:17-23.

2. Haeckel, R. (1989-1) The application of saliva in laboratory medicine, J. Clin. Chem. Clin. Biochem. 27:221-252.

3. Haeckel, R. (1989-2) Saliva, an alternative sample in clinical chemistry, JIFCC 2:208-216.

4. Borg and Birkhed (1988) Secretion of glucose in human parotid saliva after carbohydrate intake. Scand. J. Dent. Res, 96:551-556.

5. Reuterving et al. (1987) Salivary flow rate and salivary glucose concentration in patients with diabetes mellitus, Diabet. Metab. 13:457-462.

6. Zweig and Campbell (1993) “Receiver-Operating Characteristic (ROC) Plots: A Fundamental Evaluation Tool in Clinical Medicine”, Clin. Chem. 39: 561-577.

7. American Diabetes Association Position Statement: Screening for Type 2 Diabetes, Diabetes Care, 23 Supp: 1, Clinical Practice recommendations 2000.

8. Report on the Expert Committee on the Diagnosis and Classification of Diabetes Mellitus, Diabetes Care, 23 (1) Supp: S4-S19, 2000.

1. A device for sequestering a low molecular weight analyte from a saliva sample and storing the low molecular weight analyte until detection, comprising:
(a) a matrix, said matrix being able to absorb water from the sample and reversibly adsorb the low molecular weight analyte from the sample, and said matrix comprising cavities sized to accommodate the low molecular weight analyte within, and to exclude selected components of saliva, and
(b) a container, into which the saliva sample and the matrix may be placed.

2. The device of claim 1 wherein the low molecular weight analyte is glucose.

3. The device of claim 1 further comprising a preservative in the container.

4. The device of claim 1 wherein the matrix is affixed to a solid support.

5. The device of claim 1 further comprising a membrane, said membrane providing a low molecular weight analyte-permeable barrier between the sample and the matrix.

6. The device of claim 1 further comprising a means for sealing the container.

7. The device of claim 1 wherein the container contains a marking that indicates the level to which the sample must be collected.

8. The device of claim 1 further comprising a plastic dropper, for transfer of saliva into the container.

9. The device of claim 1 wherein the container is comprised of plastic or glass.

10. The device of claim 1 wherein the matrix is cross-linked agarose.

11. The device of claim 1 wherein the matrix is a zeolite.

12. The device of claim 1 wherein the matrix is one of: (a) a cross-linked anionic polyamine flocculent material, or (b) a cross-linked anionic polyacrylamide flocculent material.

13. The device of claim 1 wherein the matrix is selected from a group consisting of: cross-linked aluminosilicate, clinoptilolite, gel alumina, alumino-silicate adsorbant sieves, activated carbon, activated charcoal, cross-linked cationic polyamine flocculent material, and cross-linked cationic polyacrylamide flocculent material.

14. The device of claim 3 wherein the preservative is one of: (a) 2-methyl-4-isothiazolin-3-one, or (b) 5-chloro-2-methyl-4-isothiazolin-3-one.

15. The device of claim 3 wherein the preservative is selected from a group consisting of: sodium azide, benzoic acid, sorbic acid, salts of sorbic acid, thimerosal, phenyl mercuric acetate, phenyl mercuric nitrate, ethyl alcohol, chlorhexidine gluconate and benzalkonium chloride.

16. The device of claim 5 wherein the membrane is selected from a group consisting of: glass fiber, filter paper, PVC membrane filters, polypropylene, polyethersulfone prefilters, RW prefilters; nylon membranes, supported PTFE, unsupported PTFE, hydrophilized PTFE, quartz fiber filters, mixed cellulose esters filters, polyvinylidene fluoride membrane, dacron membranes, rayon membranes, polyethelene membranes, cellulose acetate membranes, cellulose nitrate membranes, and nitrocellulose membranes.

17. A method of sequestering a low molecular weight analyte from a saliva sample, comprising:
(b) providing:
(i) a matrix, said matrix being able to absorb water from the sample and reversibly adsorb the low molecular weight analyte from the sample, and said matrix comprising cavities sized to accommodate the low molecular weight analyte within, and to exclude selected components of saliva, and
(ii) a saliva sample, and
(b) contacting the matrix with the saliva sample.

18. A method of sequestering a low molecular weight analyte from a saliva sample and storing the low molecular weight analyte until detection, comprising:
(b) providing:
(i) a matrix, said matrix being able to absorb water from the sample and reversibly adsorb the low molecular weight analyte from the sample, and said matrix comprising cavities sized to accommodate the low molecular weight analyte within, and to exclude selected components of saliva, and
(ii) a preservative;
(b) contacting the matrix with the sample and preservative, such that the matrix becomes hydrated by the sample.

19. The method of claim 18 wherein, at step (b), the sample and the preservative are placed into a container.

20. The method of claim 18 wherein the low molecular weight analyte is glucose.

21. The method of claim 19 wherein at step (b), the sample is introduced into the container by expectoration into the container.

22. The method of claim 19 wherein at step (b), the matrix is placed into the container before the sample, and further comprising the step of placing a membrane on the top surface of the matrix before the sample is added, said membrane providing a low molecular weight analyte-permeable barrier between the sample and the matrix.

23. The method of claim 19 further comprising the step of sealing the container, after the sample has been added in step (b).

24. A method of transporting a low molecular weight analyte in a saliva sample from a location of collection to a location of detection comprising:
(a) sequestering and storing the low molecular weight analyte according to the method of claim 23, and
(b) transporting the sample to a location where it will be analysed.

25. The method of claim 24 wherein the low molecular weight analyte is glucose.

26. A method of assaying for a low molecular weight analyte in a saliva sample comprising:
(f) providing a matrix, said matrix being able to absorb water from the sample and reversibly adsorb the low molecular weight analyte from the sample, and said matrix comprising cavities sized to accommodate the low molecular weight analyte within, and to exclude selected components of saliva;
(g) contacting the matrix with the sample;
(h) removing liquid that is not absorbed into the matrix;
(i) releasing the low molecular weight analyte that is sequestered within the cavities of the matrix, and
(j) detecting the released low molecular weight analyte.

27. The method of claim 26 wherein the low molecular weight analyte is glucose.

28. A method of assaying for a low molecular weight analyte in a saliva sample comprising:
(a) sequestering and storing the low molecular weight analyte according to the method of claim 23;
(b) transporting the sample to a location where it will be analysed;
(c) removing liquid that is not absorbed into the matrix;
(d) releasing the low molecular weight analyte that is sequestered within the cavities of the matrix; and
(k) detecting the released low molecular weight analyte.

29. The method of claim 28 wherein the low molecular weight analyte is glucose.

30. The method of claim 18 further comprising the step of stimulating salivary gland secretion before step (b).

31. A kit for sequestering a low molecular weight analyte from a saliva sample and storing the low molecular weight analyte until detection, comprising:
(a) a matrix, said matrix being able to absorb water from the sample and reversibly adsorb the low molecular weight analyte from the sample, and said matrix comprising cavities sized to accommodate the low molecular weight analyte within, and to exclude selected components of saliva, and
(b) a container into which the matrix and the saliva sample may be placed.

32. The kit of claim 31 further comprising a preservative.

33. The kit of claim 32 further comprising a lid for sealing the container.

34. The device of claim 1 wherein the matrix is a first matrix and said first matrix is in the bottom of the container, further comprising a second matrix in the container, said second matrix being layered on the top surface of the first matrix, and separated from said first matrix by a membrane, and said second matrix being able to absorb water from the sample, and said second matrix comprising cavities sized to accommodate the low molecular weight analyte within, and to exclude selected components of saliva.

35. The device of claim 34 wherein the low molecular weight analyte is glucose.

36. A method of sequestering a low molecular weight analyte from a saliva sample and storing the low molecular weight analyte until detection, comprising:
(a) providing:
(i) a first matrix, said first matrix being able to absorb water from the sample and reversibly adsorb the low molecular weight analyte from the sample, and said first matrix comprising cavities sized to accommodate the low molecular weight analyte within, and to exclude selected components of saliva;
(ii) a second matrix, and said second matrix being able to absorb water from the sample and said second matrix comprising cavities sized to accommodate the low molecular weight analyte within, and to exclude selected components of saliva, and
(iii) a preservative;
(b) placing said first matrix at the bottom of a container, and layering said second matrix on the top surface of the first matrix,
(c) introducing the sample into the container by applying the sample to the top surface of second matrix; and
(d) allowing the sample to hydrate the second matrix and then the first matrix.

37. The method of claim 36 wherein the low molecular weight analyte is glucose.

38. The method of claim 36 wherein at step (b) a membrane is placed between the first matrix and the second matrix.

39. The method of claim 36 wherein at step (c), the sample is introduced into the container by expectoration into the container.

40. The method of claim 36 further comprising the step of placing a membrane on the top surface of the second matrix before the sample is added in step (c), said membrane providing a low molecular weight analyte-permeable barrier between the sample and the matrix.

41. The method of claim 36 further comprising the step of sealing the container, after the sample has been added in step (b).

42. A method of transporting a low molecular weight analyte in a saliva sample from a location of collection to a location of detection comprising:
(a) sequestering and storing the low molecular weight analyte according to the method of claim 41, and
(b) transporting the sample to a location where it will be analysed.

43. A method of assaying for a low molecular weight analyte in a saliva sample comprising:
(a) providing:
(i) a first matrix, said first matrix being able to absorb water from the sample and reversibly adsorb the low molecular weight analyte from the sample, and said first matrix comprising cavities sized to accommodate the low molecular weight analyte within, and to exclude selected components of saliva;
(ii) a second matrix, and said second matrix being able to absorb water from the sample and said second matrix comprising cavities sized to accommodate the low molecular weight analyte within, and to exclude selected components of saliva,
(b) contacting the sample with the second matrix and then with the first matrix;
(c) removing liquid that is not absorbed into the first or the second matrix;
(d) releasing the low molecular weight analyte that is sequestered within the cavities of the first matrix, and
(e) detecting the released low molecular weight analyte.

44. The method of claim 43 wherein the low molecular weight analyte is glucose.

45. A method of assaying for a low molecular weight analyte in a saliva sample comprising:
(a) sequestering and storing the low molecular weight analyte according to the method of claim 41;
(b) transporting the sample to a location where it will be analysed;
(c) removing liquid that is not absorbed into the first or the second matrix;
(d) releasing the low molecular weight analyte that is sequestered within the cavities of the first matrix; and
(e) detecting the released low molecular weight analyte.

46. The method of claim 45 wherein the low molecular weight analyte is glucose.

47. A device for sequestering a low molecular weight analyte from a saliva sample and storing the low molecular weight analyte until detection, comprising:
(a) a matrix, said matrix being able to absorb water from the sample and reversibly adsorb the low molecular weight analyte from the sample, and said matrix comprising cavities sized to accommodate the low molecular weight analyte within, and to exclude selected components of saliva, and
(b) a solid support to which said matrix is affixed.

48. The device of claim 47 in which the solid support is selected from the group consisting of: stick, rod, slide, wick and card.

49. The device of claim 48 further comprising a container, into which one of (a) the sample and (b) the matrix, may be placed.

50. The device of claim 48 further comprising a container into which both the sample and the matrix may be placed.

51. The device of claim 47 wherein the low molecular weight analyte is glucose.

52. The device of claim 47 further comprising a preservative on the support, in the matrix, or both.

53. The device of claim 47 wherein the matrix is cross-linked agarose.

54. The device of claim 47 wherein the matrix is a zeolite.

55. The device of claim 47 wherein the matrix is one of: (a) a cross-linked anionic polyamine flocculent material, or (b) a cross-linked anionic polyacrylamide flocculent material.

56. The device of claim 47 wherein the matrix is selected from a group consisting of: cross-linked aluminosilicate, clinoptilolite, gel alumina, alumino-silicate adsorbant sieves, activated carbon, activated charcoal, cross-linked cationic polyamine flocculent material, and cross-linked cationic polyacrylamide flocculent material.

57. The device of claim 52 wherein the preservative is one of: (a) 2-methyl-4-isothiazolin-3-one, or (b) 5-chloro-2-methyl-4-isothiazolin-3-one.

58. The device of claim 52 wherein the preservative is selected from a group consisting of: sodium azide, benzoic acid, sorbic acid, salts of sorbic acid, thimerosal, phenyl mercuric acetate, phenyl mercuric nitrate, ethyl alcohol, chlorhexidine gluconate and benzalkonium chloride.
